(19) United States
(12) Patent Application Publication
(10) Pub. No.: US 2021/0225494 A1
(43) Pub. Date: Jul. 22, 2021

(54) SYSTEMS AND METHODS OF MANAGING AND REGULATORY COMPLIANCE

(71) Applicants: Tony Baker, Houston, TX (US); Melinia Jackson, Houston, TX (US)

(72) Inventors: Tony Baker, Houston, TX (US); Melinia Jackson, Houston, TX (US)

(21) Appl. No.: 16/749,619

(22) Filed: Jan. 22, 2020

(51) Int. Cl. G16H 40/20; G06Q 40/00

(52) U.S. Cl. CPC G16H 40/20; G06Q 40/12

(57) ABSTRACT

The present disclosure comprises a method for managing event data. The method can include receiving an event comprising event data, at a processor, wherein the event data comprises a receipt image from a vendor associated with the event. The method can include generating, at a processor, a unique identifier associated with the event data. The method can comprise determining at a processor, at least one healthcare provider associated with the event. The method can comprise transmitting an email comprising the event data to at least one of: a healthcare provider associated with the event, an assistant to the healthcare provider, the at least one representative, and a vendor associated with the event. The method can comprise storing, in a memory, in association with the unique identifier, the event data.

### TECHNICAL FIELD

The present disclosure relates generally to the field of data reporting, and more particularly, to reporting management systems and methods for regulatory compliance.

### BACKGROUND OF THE INVENTION

Since the Physician Payments Sunshine Act (the “Sunshine Act”) became law as part of the Affordable Care Act, general payments made to physicians and teaching hospitals from pharmaceutical/medical device companies (those that include meals) have totaled nearly 61 million (˜$15.2 Billion) reported.

However, vulnerabilities still exist between the accuracy of information reported, the timing of entries, and the narrow window physicians have to appeal inaccurate payments. The Sunshine Act stipulates that physicians have 45 days to dispute all gifts rendered, before it becomes public knowledge—of the millions of payments filed since reporting was mandated in 2013, less than 1/10 of a percent have been disputed. This vulnerability has led many physicians to decrease or eliminate the number of marketing interactions with pharmaceutical/medical device representatives in exchange for preserving reputations among their patients and colleagues. Thus, there exists a need for a solution that addresses the management of data and the dispute process by providing the evidence of all transactions reported in the name of the physician in real-time.

### SUMMARY OF THE INVENTION

The present disclosure meets the needs described above comprising a method and system for data reporting and management. The method can comprise receiving an event comprising event data, at a processor. The event data can include a receipt image from a vendor associated with the event. A unique identifier associated with the event data can be generated. The method can comprise determining by the processor, at least one healthcare provider associated with the event. The method can comprise transmitting a message via email; for example, comprising the event data to at least one of: the healthcare provider associated with the event, an assistant to the healthcare provider, at least one representative, and a vendor associated with the event. The method can also comprise storing, in a memory, in association with the unique identifier, the event data.

In another embodiment, the disclosure can comprise a system that includes a memory having computer-executable instructions encoded thereon and a processor functionally coupled to the memory. The processor can be configured, by the computer-executable instructions, for receiving an event comprising event data, at a processor. The event data can include a receipt image from a vendor associated with the event. A unique identifier associated with the event data can be generated. The processor can determine at least one healthcare provider associated with the event. The processor can transmit a message such as an email comprising the event data to at least one of: a healthcare provider associated with the event, an assistant to the healthcare provider, the at least one representative, and a vendor associated with the event. The memory, in association with the unique identifier, can store the event data.

In another aspect, a non-transitory computer-readable medium can have computer-executable instructions encoded thereon. When executed, the computer-executable instructions can cause a computer to perform steps comprising receiving an event comprising event data, at a processor. The event data can include a receipt image from a vendor associated with the event. A unique identifier associated with the event data can be generated. The processor can determine at least one healthcare provider associated with the event. The processor can transmit an email comprising the event data to at least one of: a healthcare provider associated with the event, an assistant to the healthcare provider, the at least one representative, and a vendor associated with the event. The memory, in association with the unique identifier, can store the event data.

In view of the foregoing, it will be appreciated that the present disclosure provides improved data reporting and management. The specific systems and techniques for accomplishing the advantages described above will become apparent from the following detailed description of the embodiments and the appended drawings and claims.

### BRIEF DESCRIPTION OF THE DRAWINGS

FIG. 1 depicts a sample system of the disclosure.

FIG. 3 depicts a block diagram of the method for capturing event data.

FIG. 3 depicts a flowchart of an embodiment of the method depicted in FIG. 2.

FIG. 4 depicts a dashboard interface of the representative profile.

FIG. 5 depicts a dashboard interface to enter healthcare provider data.

FIG. 6 depicts a dashboard interface to enter restaurant/vendor data.

FIG. 7 depicts a dashboard interface to enter receipt data.

FIG. 8 depicts a dashboard interface showing a representative records.

FIG. 9 depicts a dashboard interface showing a monthly report for a representative.

FIG. 10 depicts a dashboard showing a report summarizing representative activity for a district manager.

### DETAILED DESCRIPTION

Illustrative embodiments of the disclosure will now be described more fully hereinafter with reference to the accompanying drawings, in which some, but not all embodiments of the disclosure are shown. The disclosure may be embodied in many different forms and should not be construed as limited to the embodiments set forth herein; rather, these embodiments are provided so that this disclosure will satisfy applicable legal requirements.

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.”

The following description is provided as an enabling teaching of the disclosed articles, systems, and methods in their best, currently known embodiments. To this end, those skilled in the relevant art will recognize and appreciate that many changes can be made to the various aspects of the articles, systems, and methods described herein, while still obtaining the beneficial results of the disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “a gasket” can include two or more such gaskets unless the context indicates otherwise.

As used throughout, “substantially” with respect to a measure can refer to a range of values comprising +/−10 degrees. For example, substantially orthogonal, normal, or parallel can include embodiments, where the referenced components are oriented +/−10 degrees of being classified as orthogonal, normal, or parallel respectively.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

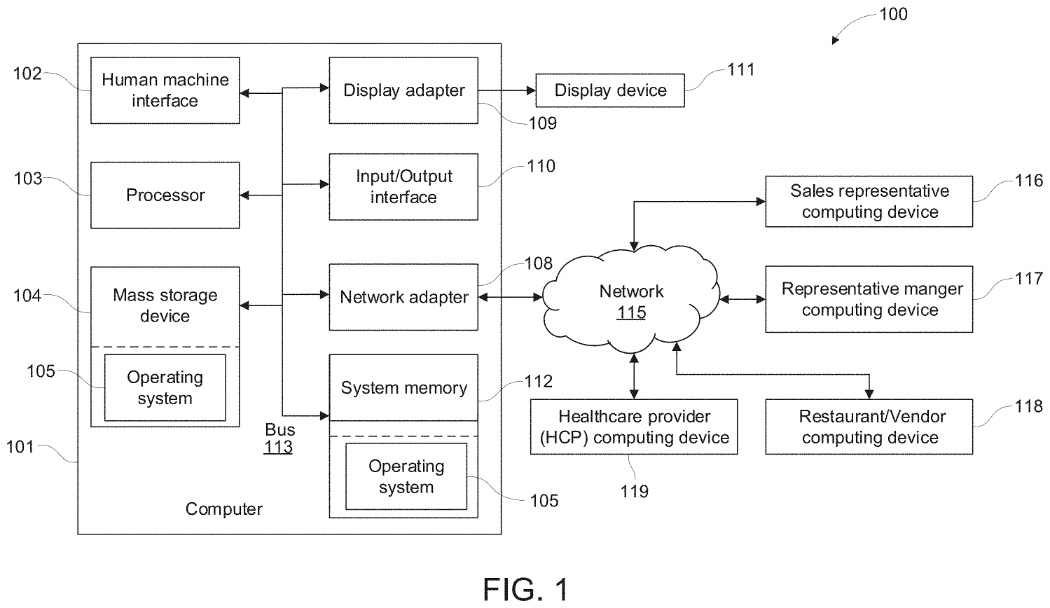

In an exemplary aspect, the methods and systems can be implemented on a computer 101 as illustrated in FIG. 1 and described below. By way of example, the system 100 of FIG. 1. Similarly, the methods and systems disclosed can utilize one or more computers to perform one or more functions in one or more locations. FIG. 1 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, and mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that performs particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 101. The components of the computer 101 can comprise, but are not limited to, one or more processors 103, a system memory 112, and a system bus 113 that couples various system components including the one or more processors 103 to the system memory 112. The system can utilize parallel computing.

The system bus 113 represents one or more of several possible types of bus structures, including a memory bus or memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 113, and all buses specified in this description can also be implemented over a wired or wireless network connection and each of the subsystems, including the one or more processors 103, a mass storage device 104, an operating system 105, a network adapter 108, the system memory 112, an Input/Output Interface 110, a display adapter 109, a display device 111, and a human machine interface 102, can be contained within one or more remote computing devices 116-119 at physically separate locations, connected through buses of this form, in effect implementing a fully distributed system.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM).

In another aspect, the computer 101 can also comprise other removable/non-removable, volatile/non-volatile computer storage media. By way of example, FIG. 1 illustrates the mass storage device 104 which can provide non-volatile storage of computer code, computer readable instructions, data structures, program modules, and other data for the computer 101. For example and not meant to be limiting, the mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like. The data for the events can be stored in the mass storage device 104 in any of one or more databases known in the art. Examples of such databases comprise, DB2®, Microsoft® Access, Microsoft® SQL Server, Oracle®, MySQL, PostgreSQL, and the like. The databases can be centralized or distributed across multiple systems. Optionally, any number of program modules can be stored on the mass storage device 104, including by way of example, the operating system 105 and/or an Optical Character Recognition (OCR) module.

In another aspect, the user can enter commands and information into the computer 101 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 103 via the human machine interface 102 that is coupled to the system bus 113, but can be connected by other interface and bus structures, such as a parallel port, game port, an IEEE 1394 Port (also known as a Firewire port), a serial port, or a universal serial bus (USB).

In yet another aspect, the display device 111 can also be connected to the system bus 113 via an interface, such as the display adapter 109. It is contemplated that the computer 101 can have more than one display adapter 109 and the computer 101 can have more than one display device 111. For example, a display device can be a monitor, an LCD (Liquid Crystal Display), or a projector. Through the display the device, the user can view a dashboard, user interface portal. In addition to the display device 111, other output peripheral devices can comprise components such as speakers (not shown) and a printer (not shown) which can be connected to the computer 101 via the Input/Output Interface 110. Any step and/or result of the methods can be output in any form to an output device. Such output can be any form of visual representation, including, but not limited to, textual, graphical, animation, audio, tactile, and the like. The display 111 and computer 101 can be part of one device, or separate devices.

The computer 101 can operate in a networked environment using logical connections to one or more remote computing devices 116, 117, 118, and 119. By way of example, a remote computing device can be a personal computer, portable computer, smartphone, a server, a router, a network computer, a peer device, or other common network node, and so on. Logical connections between the computer 101 and remote computing devices 116, 117, 118, and 119 can be made via a network 115, such as a local area network (LAN) and/or a general wide area network (WAN), or the connection may be made to an external computer (for example, through the Internet using an Internet Service Provider). Transmission of program code embodied on a computer readable medium can occur using any appropriate medium including, but not limited to, wireless, wired, optical fiber cable, radio frequency (RF), or any suitable combination thereof.

In an example embodiment, the first remote computing device 116 can be associated with the sales representative. The second remote computing device 117 can be associate with sales representative manager. The third remote computing device 118 can be associated with the restaurant/vendor. The fourth remote computing device 119 can be associated with the healthcare provider HCP. Such network connections can be through the network adapter 108. The network adapter 108 can be implemented in both wired and wireless environments. Such networking environments are conventional and commonplace in dwellings, offices, enterprise-wide computer networks, intranets, and the Internet.

For purposes of illustration, application programs and other executable program components such as the operating system 105 are illustrated herein as discrete blocks, although it is recognized that such programs and components reside at various times in different storage components of the computing device 101, and are executed by the one or more processors 103 of the computer. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

As shown in FIG. 2, the method 200 can be represented as a block diagram. The method 200 can initiate at block 202 which comprises receiving an event comprising event data. In a further aspect, receiving the event can comprise receiving attendance data associated with the event data. The method 200 can proceed to block 204 comprising generating a unique identifier associated with the event data. The method 200 can proceed to block 206 which comprises determining at least one healthcare provider associated with the event. In a further aspect, determining the at least one healthcare provider can comprise: parsing the receipt image into time data and transaction data for the event; and assigning transaction data to the healthcare provider wherein transaction data comprises: a vendor item; cost of the vendor item; and the healthcare provider associated with the vendor item. The method 200 can proceed to block 208 comprising storing, in a memory, in association with the unique identifier, the event data.

The method 200 can proceed to block 210 comprising generating a first-type alert based on the transaction data and transmitting the first-type alert to a representative manager or at least one representative. In a further aspect, generating the first-type alert can include comparing the transaction data to a first threshold value associated with the at least one representative. The method can proceed to block 212 comprising generating a second-type alert based on the time data and attendance data of the event. Generating the second-type alert can include comparing the auxiliary time data and the auxiliary attendance data of an auxiliary event with the time data and the attendance data of the event, and determining that the comparison of the time data and attendance data impacts a second threshold.

The method 200 can proceed to block 214 that comprises transmitting a reporting email comprising the event data to at least one of: a healthcare provider associated with the event, an assistant to the healthcare provider, the at least one representative, and a vendor associated with the event. The method 200 can proceed to block 216, which comprises updating the event data based on receiving a response to the email transmitted to the healthcare provider. The response from at least one of the initial recipients can be used to update the original event data. The response can comprise supplemental event data associated with the event.

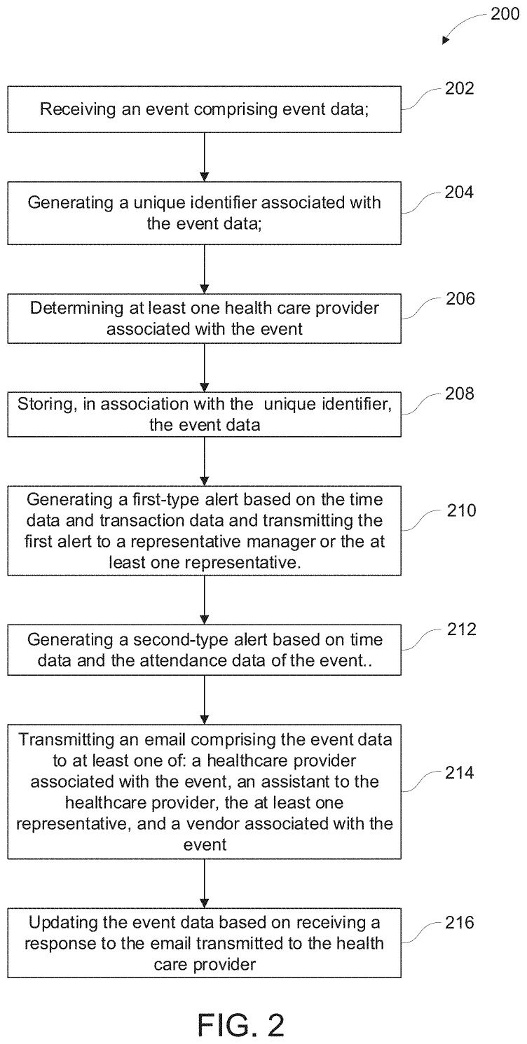

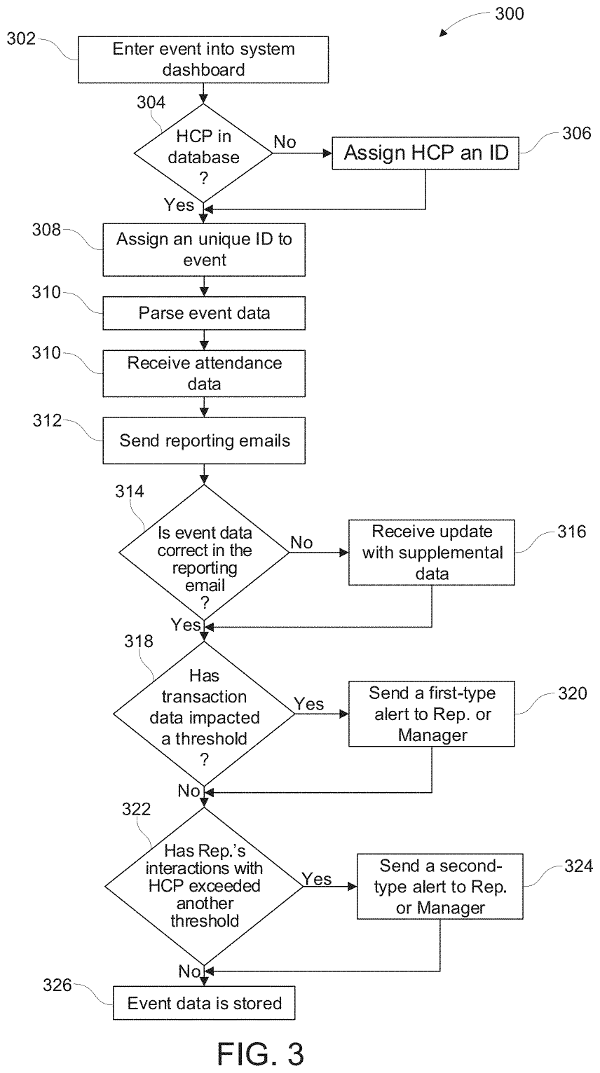

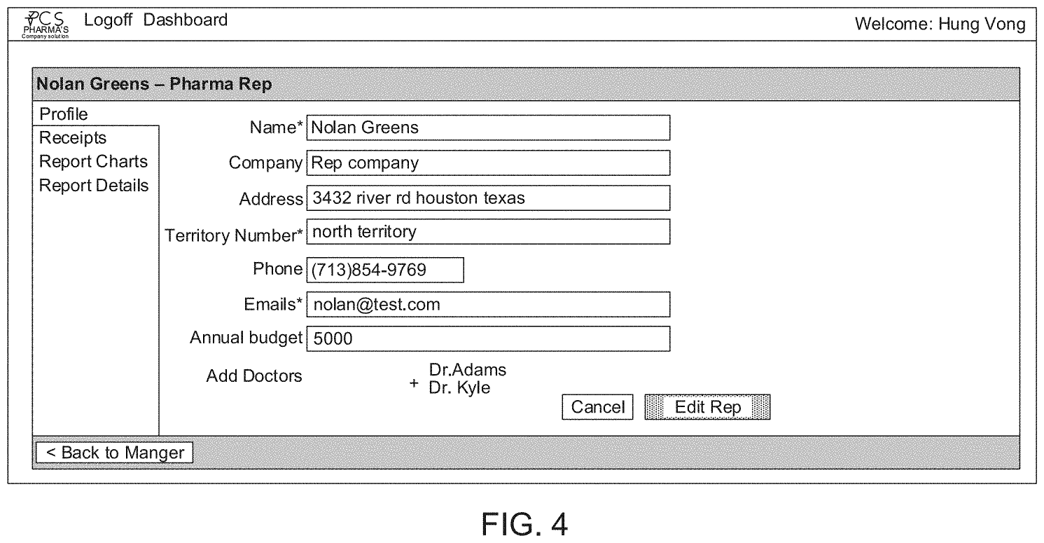

As shown in FIG. 3, the flowchart 300 depicts an example embodiment of the method 200 for reporting data and remaining in compliance with the Sunshine Act. The operations of FIG. 3 may be performed by the system in FIG. 1 (e.g., in parallel, in series, or in any suitable order). The system 100 can initiate a process at step 302, wherein the representative or the representative manager enters the event into the system. As shown in FIG. 4, a dashboard user interface for adjusting the company representative profile is shown. Accordingly, the representative can access their profile via the dashboard. As shown in this view, the representative can initially enter or update information. In addition to personal information, the representative can enter their allocated budget and any healthcare providers (HCPs) that the representative is associated.

The event can refer to any activity involving the interaction between a company representative or manager and the healthcare provider. For example, the event can be a catered function by a vendor at the healthcare provider's facility or at an off-site location. In a further aspect, the event can be a breakfast, lunch, brunch, dinner, coffee, or cocktails at a restaurant or establishment. In yet a further aspect, the event can involve any occurrence involving an exchange of monetary value provided to the healthcare provider.

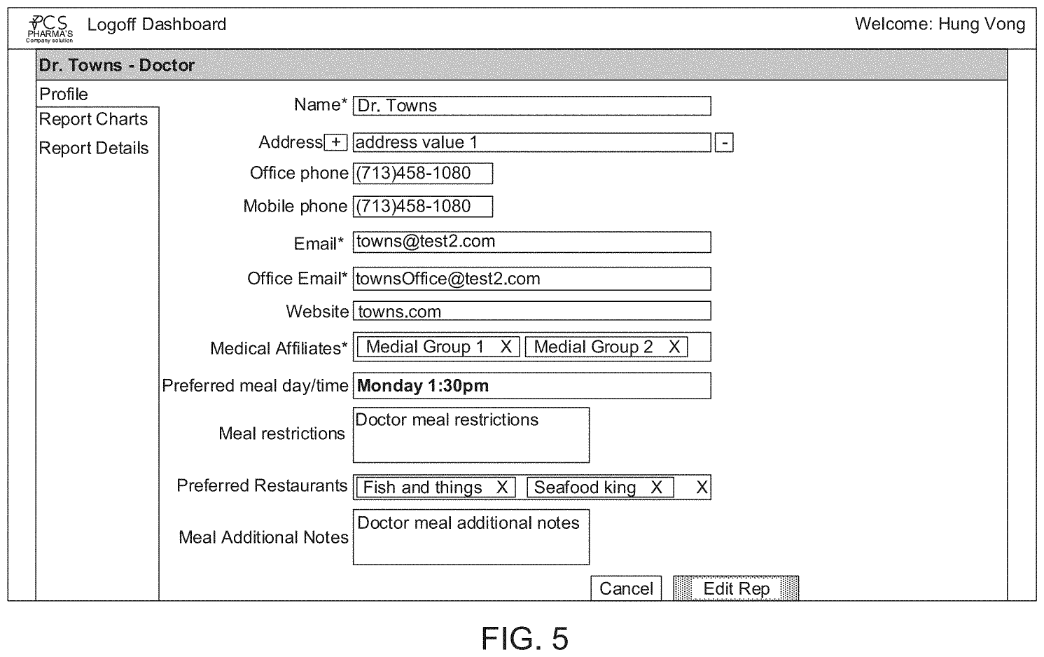

After the event is entered into the system interface, the computer can proceed to decision step 304 wherein a determination is made whether the healthcare provider is currently in the database. If the healthcare provider is not currently in the database, the computer can proceed to step 306. In step 306, the healthcare provider can be assigned an identifier for subsequent entries related to that healthcare provider. For instance, where the healthcare provider has not been previously identified, the processor can generate a dashboard display as shown in FIG. 5. The dashboard allows the representative or manager entering the data to provide the contact information for the healthcare provider including but not limited to: the healthcare provider name, assistant, email, phone numbers, medical affiliates, address, and meal preferences. Upon entrance of the healthcare provider information, the system can allocate an a code, such as a truncated name, full name, or alphanumeric combination, to the healthcare provider for use in the current or subsequent unique identifier generation.

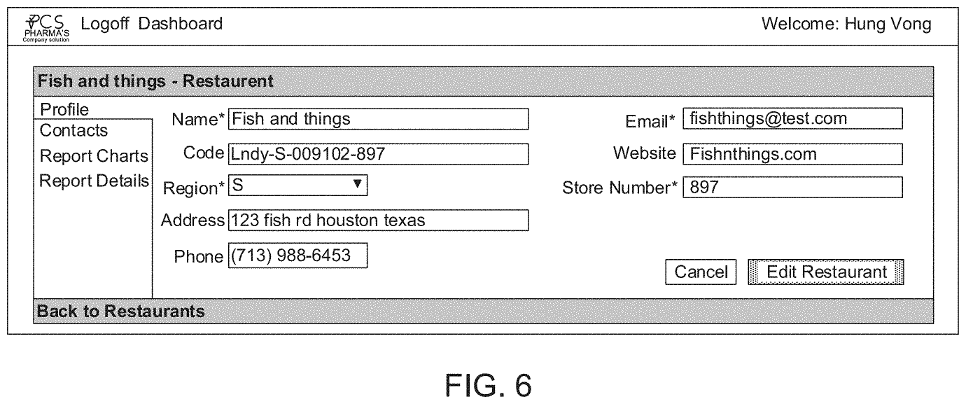

In an alternative embodiment, the computer can process a decision step that determines whether a restaurant/vendor data needs to be entered. When the restaurant/vendor has not been previously identified, the processor can generate a display of the dashboard view as shown in FIG. 6. As shown in FIG. 6, the dashboard can allow the representative to enter the vendor name, region, address, phone number, email, and website and store number. After the restaurant/vendor has been entered, the system can also generate an identifier (code) for the vendor. For example, the ID can be based on a combination of the name of the restaurant/vendor, geographic region, the date the restaurant becomes a member in the system and a store number.

In referring back to decision step 304, if the healthcare provider is currently in the database, the computer can proceed to step 308 wherein a unique identifier can be assigned to the event. In particular, the unique identifier would be attached to each event entered into the system memory and/or storage device. In a further aspect, various combinations are possible to generate the unique identifier. In one aspect, these combinations could be based on coded portions of data associated with the event such as the vendor, date, HCP, and/or representative. In another aspect, the unique identifier can be an alphanumeric key that is randomly generated.

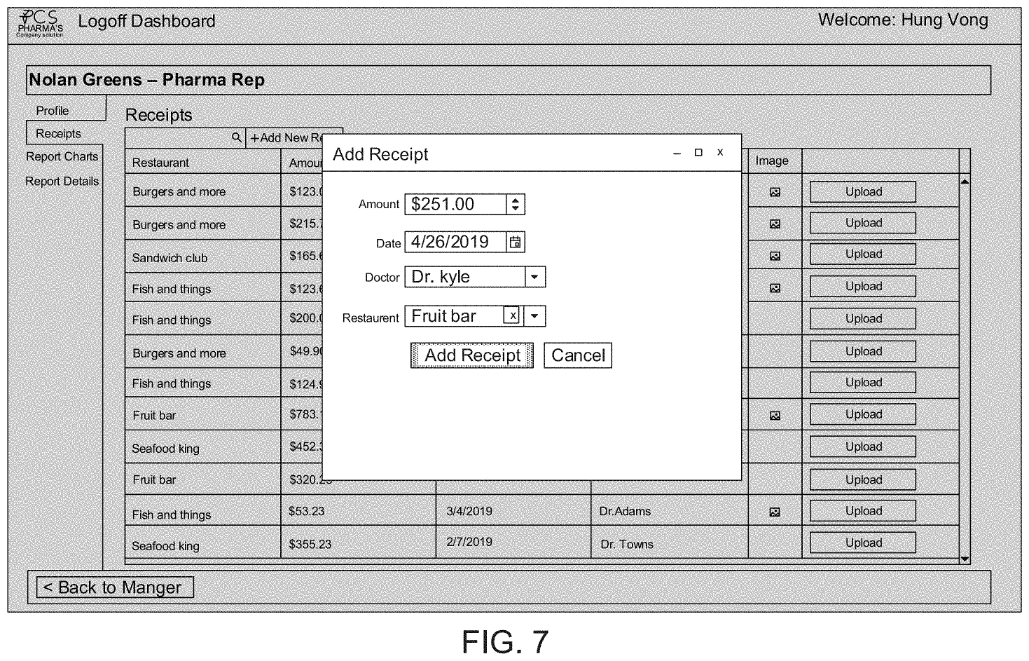

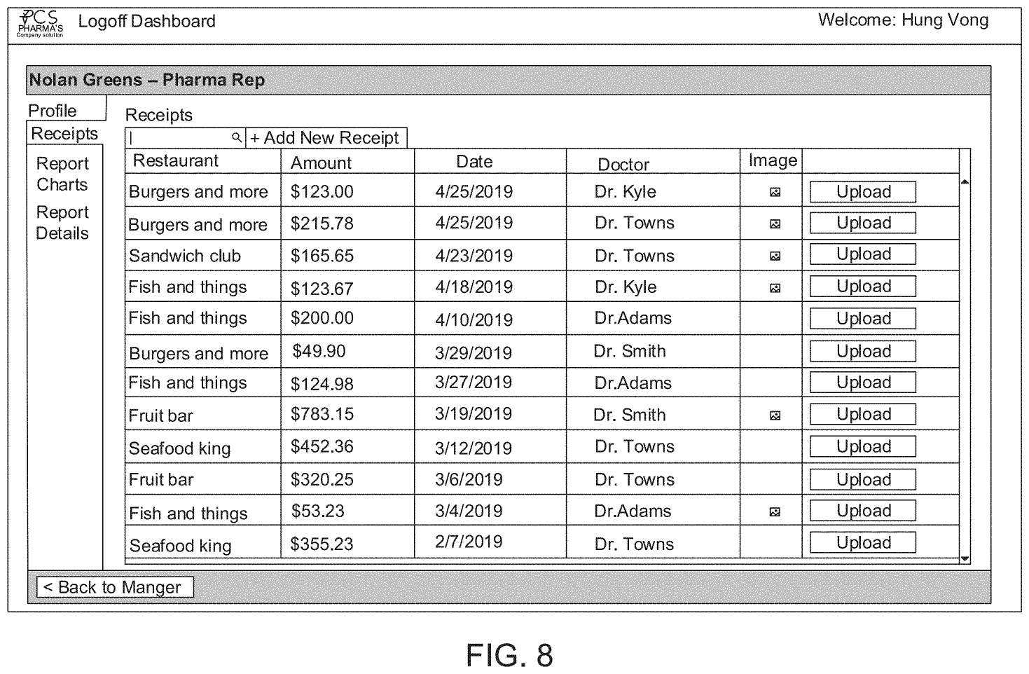

After the unique identifier has been assigned to the event, the computer can proceed to step 310, wherein the event data received can be parsed. As shown in FIG. 7, the processor can initiate a pop-up menu through the dashboard, where the representative can enter transaction data such as the event cost, date, restaurant, and healthcare provider (HCP). Once the data is entered, the data can be stored in the respective field to a database in the storage device, and associated with the unique identifier. The representative can then select the “Add Receipt” button to upload an image of the receipt. The picture of the receipt may be saved as an image file. In the instance where multiple HCPs are in attendance, the processor can default to evenly split the event's cost across each of the HCPs. Further as shown in FIG. 8, the representative's (Nolan Greens as shown in the FIG) activity can be accessed through the dashboard by the representative or individuals with the allotted permissions, such as the representative's manager. In a further aspect, the dashboard views can be configured with text entry fields, associated with database field, in the instance where an anticipated selection is not available in a drop down menu.

In an alternate embodiment, the computer can receive the image file and parse the necessary data from the image file. In particular, the processor can use an Optical Character Recognition (OCR) software module to recognize the pertinent data from the receipt. Through parsing the image data, the processor can identify characters related to certain data fields. As a further example, the OCR module can recognize a pattern of digits to be representative of a price, address or telephone number. Similarly, a pattern of letters can be used to recognize a name, such as the vendor, a vendor item, such as a food item or drink/cocktail, or a healthcare provider.

After parsing the data, the computer can proceed to step 310, wherein the computer receives attendance data for the event. The attendance data can comprise all of the attendees that were transferred a monetary benefit in addition to the representative. The attendance data can be entered directly through the user dashboard by a representative or manager as shown in FIG. 7. In another embodiment, the attendance data can be determined using the OCR software, such as when representative may have written the attendees on the receipt.

After the attendance data is received, the computer can proceed to step 312, where a reporting email can be sent to the healthcare provider, the assistant to the healthcare provider, the representative, and the vendor. The reporting email can provide a measure of proof to be in compliance with the Sunshine Act. Each of the reporting emails and receipts can be stored in the database. In one aspect, the email can contain information such as the date of the event, the vendor, and the cost of the event. In another aspect, the processor can generate a summary page that indicates details all of the event data as a visual display to view. The reporting email can be sent to the representative, healthcare provider, assistant to the healthcare provider and the vendor. In a further aspect, the representative manager can also receive an email. In a further aspect, the representative, can identify the data field of event data that can be sent to each recipient of the reporting email. For example, the vendor can receive an email comprising: event time, event cost and company representative who paid.

The event data can include time data, such as the date of the event, the time of the event. In a further aspect, the duration of the event can be entered. The transaction data can include: the cost of the event, vendor associated with the event, the items purchased at the event, and the items associated with each healthcare provider who attended the event. In yet a further example, if the event is a catered event, the transaction data may include the main healthcare provider associated with the event. The attendance data can include all attendees to the event.

After the reporting emails are sent, the computer can proceed to decision step 314, wherein it is determined if all of the entered information is correct in the reporting email. If the entered information is not correct, the computer can be configured to receive the correct information and proceed to step 316. At step 316, the representative, representative manager, vendor/restaurant, or HCP can correct the original event data including the time data and transaction data by providing supplemental data. Referring back to step 314, if the event data is correct, the computer can proceed to step 318.

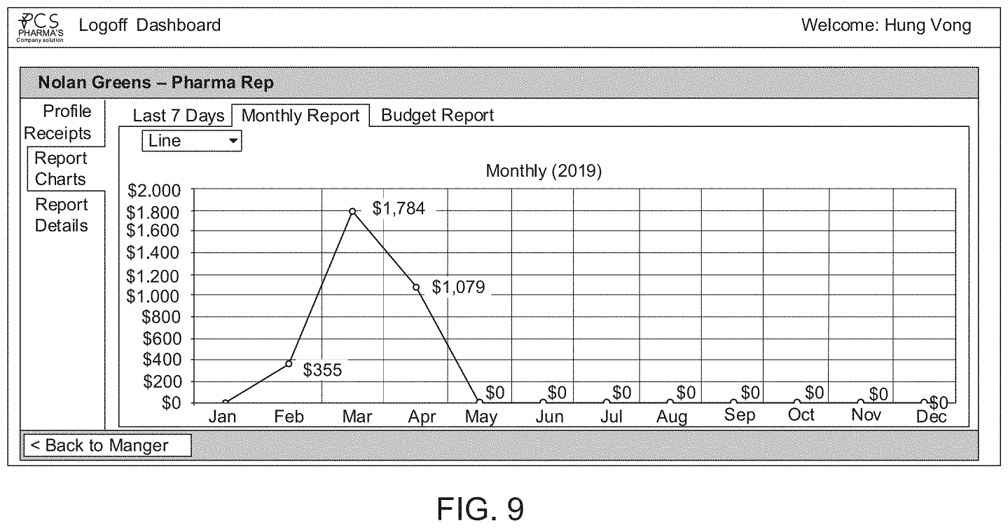

At decision step 318, the computer can determine whether a first-type alert needs to be generated. The first-type alert can be an alert triggered when the representative's activity impacts thresholds set for their hosting budget. The determination can be based on whether activity has exceeded or not exceeded a particular threshold. For example, exceeding a maximum threshold may trigger an alert. Similarly, if the representative's activity does not meet a minimum threshold the alert can be triggered. As a further illustration, the representative company can allocate a certain amount of a money to a representative's hosting budget. As shown in FIG. 4, the representative has an annual hosting budget to be used. FIG. 9 shows the spending for a representative for the calendar year 2019. Based on the transaction data received associated with an event, a determination can be made based on the transaction data's impact on the hosting budget. Here, the representative may have a $1,500 monthly allocation (threshold) for hosting clients, but spent $1,784; the computer can then proceed to step 320. At step 320, the computer may send a first-type alert in the month of March because the $1,500 figure was exceeded.

In another embodiment of decision step 318, the representative may also be working under a minimum spending quota provision (e.g. $500), such that the spending in February would also trigger the first-type alert. In yet a further aspect, the first-type alert can be based on the type of item that is being hosted by the representative. For example if the transaction data indicates that the majority of the budget is being allocated to drinks/cocktails and/or gifts as opposed to food, then the first-type alert can be sent to the representative or the representative manager. These settings can be managed by the representative or manager within the dashboard to set the threshold for the proper alert. In a further aspect, the various combinations of the data points in the time data and transaction data can be used to determine analytics for visual display, such as in FIG. 9. In yet a further aspect, an alternative alert can be generated and sent when the representative's activity is in proximity to a threshold. For example, a setting can be entered into the computer to trigger when the activity is at an entered percentage, (75%, 90%, 95%, etc.) of a threshold.

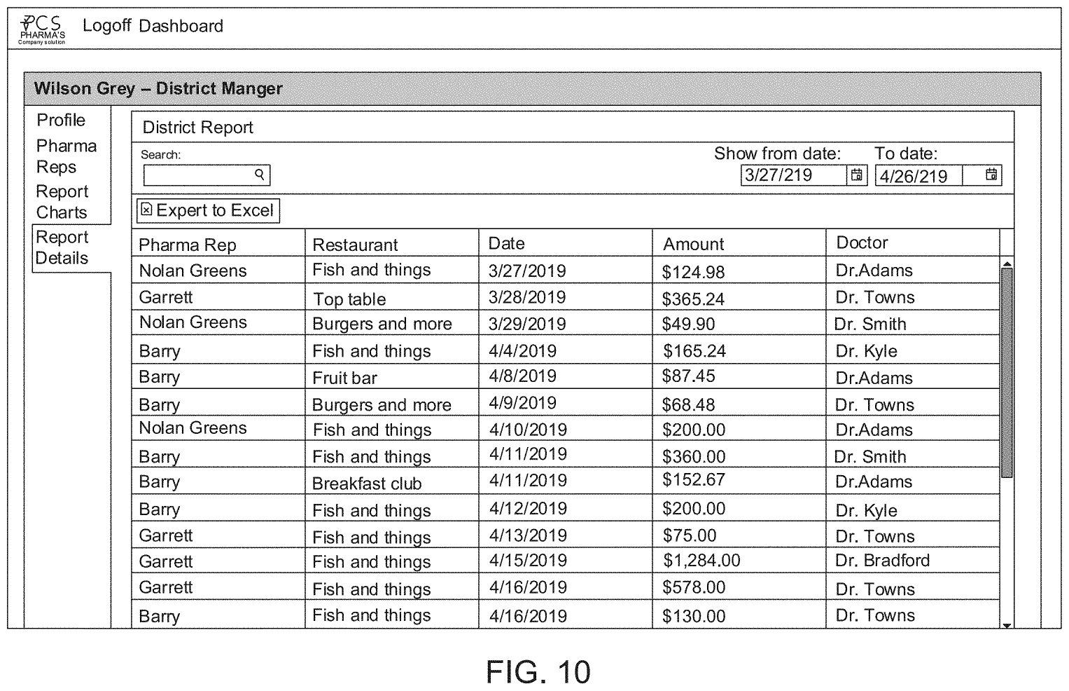

It is further considered that a first-type and/or second-type alert can be sent out as an SMS message, text message, email, or an automated phone call to the representative or representative manager. In each of these instances, the alert can be based on a threshold value set by the managing team. For example, as shown in FIG. 10, the representative manager can view the activities of their team for a date range. For instance, where a representative triggered an alert for not meeting the minimum quota, that respective alert can prompt the manager to reallocate the budget for the representative. In a further aspect, the processor can be configured such that a single alert or a series of alerts can prompt the system to reallocate budget to other team members. The reallocation can then be subsequently approved by the manager.

In referring back to decision step 318, if it is determined that representative activity has not impacted a threshold, the computer can proceed to decision step 322. At decision step 322, a decision step can be made whether the representative has had interactions with a healthcare provider that has exceeded another threshold. If the event data indicated that the company or representative's activity has exceeded another threshold, then the computer can proceed to step 324. At step 324, the second-type alert to the representative is triggered. The computer can analyze whether the representative has met with the healthcare provider multiple times in a restricted period. The number of instances and/or restricted period can be set and managed as distinct thresholds, which can both trigger the second-type of alert.

For example, based on the attendance data and the time data, the system can determine efficiencies in meeting with healthcare providers. The second-type alert can be triggered based on comparing auxiliary time data and auxiliary attendance data to current event data comprising time data and attendance data. As shown in FIG. 8, Nolan Greens met with Dr. Adams on Mar. 27, 2019 and spent $124.98. Second, an auxiliary event between the representative and Nolan Greens and Dr. Adams occurred on Apr. 10, 2019 where $200.00 was spent. The span of the period between the original event and the auxiliary event can be a threshold set by the management team. Accordingly, the processor can make a determination that the time period between the first event and the auxiliary event is too short, which can trigger the second-type alert.

Similarly, the processor can make a determination, to trigger the second type of alert based on the total amount of $324.98 being spent on Dr. Adams during this period. The amount to be spent on a HCP during the period can be a complementary threshold that can be adjusted and managed. This complementary threshold can be classified as such because of the dependency on the representative, first exceeding the number of meetings in a restricted period. In another aspect, the amount spent on the doctor during a period can be an independent threshold.

The second-type of alert can serve as a measure to prevent the representative from over allocating budget to a particular healthcare provider. In another example, Nolan Greens met with Dr. Adams on Mar. 27, 2019 and spent $124.98, while a second representative Barry met Dr. Adams on Apr. 8, 2019 and spent $87.45. The combined activity of Nolan and Barry can also trigger a second-type alert to the managing representative, Wilson Grey. The second-type alert can be triggered because of: 1) the amount spent by the company on Dr. Adams in a predefined period or 2) the number of instances that the company met with Dr. Adams in the same predefined period.

In a further aspect, the computer can provide attendance data and/or calendar reminders to representatives and/or representative managers as an additional preventative measure to over allocating budget to particular healthcare providers. Referring back to step 322, if the computer determines that the representative or company interaction's has not exceeded thresholds related to the second-type alert, the computer can proceed to step 326. At step 326, the event data is stored in the database and the process embodied in the flowchart is terminated by the computer.

The disclosure is described above with reference to block and flow diagrams of systems, methods, apparatus, and/or computer program products according to example embodiments of the disclosure. It will be understood that one or more blocks of the block diagrams and flow diagrams and combinations of blocks in the block diagrams and flow diagrams, respectively, can be implemented by computer-executable program instructions. Likewise, some blocks of the block diagrams and flow diagrams can not necessarily need to be performed in the order presented, or can not necessarily need to be performed at all, according to some embodiments of the disclosure.

These computer-executable program instructions can be loaded onto a general-purpose computer, a special purpose computer, a processor or other programmable data processing apparatus to produce a particular machine, such that the instructions that execute on the computer, processor or other programmable data processing apparatus create means for implementing one or more functions specified in the flow diagram block or blocks. These computer program instructions can also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, embodiments of the disclosure can provide for a computer program product, comprising a computer usable medium having a computer-readable program code or program instructions embodied therein, said computer-readable program code adapted to be executed to implement one or more functions specified in the flow diagram block or blocks. The computer program instructions can also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special purpose, hardware-based computer systems that perform the specified functions, elements or steps, or combinations of special purpose hardware and computer instructions.

While the disclosure has been described in connection with what is presently considered to be the most practical and various embodiments, it is to be understood that the disclosure is not to be limited to the disclosed embodiments, but on the contrary, is intended to cover various modifications and equivalent arrangements comprised within the spirit and scope of the appended claims.

What is claimed is:

1. A method, comprising:
receiving an event comprising event data, at a processor, wherein the event data comprises a receipt image from a vendor associated with the event;
generating, at the processor, a unique identifier associated with the event data;
determining at the processor, at least one healthcare provider associated with the event;
transmitting an email comprising the event data to at least one of: a healthcare provider associated with the event, an assistant to the healthcare provider, at least one representative, and a vendor associated with the event; and
storing, in a memory, in association with the unique identifier, the event data.

2. The method of claim 1, where in receiving the event comprises receiving attendance data associated with the event data;

3. The method of claim 1, wherein determining the at least one healthcare provider comprises:
parsing the receipt image into time data and transaction data; and
assigning the transaction data to the healthcare provider wherein the transaction data comprises: a vendor item; cost of the vendor item; and the healthcare provider associated with the vendor item.

4. The method of claim 3, further comprising generating a first-type alert based on the transaction data and transmitting the first-type alert to a representative manager or the at least one representative.

5. The method of claim 4, wherein generating the first-type alert comprises comparing the transaction data to a first threshold value associated with the at least one representative.

6. The method of claim 2, further comprising generating a second-type alert based on time data and the attendance data of the event.

7. The method of claim 6, wherein generating the second-type alert comprises:
comparing auxiliary time data and auxiliary attendance data of an auxiliary event with the time data and the attendance data of the event;
determining that a comparison of the time data and the attendance data exceeds a second threshold.

8. The method of claim 1, further comprising updating the event data based on receiving a response to the email transmitted to the healthcare provider.

9. A system, comprising:
a memory having computer-executable instructions encoded thereon; and
a processor functionally coupled to the memory and configured by the computer-executable instructions to:
receiving an event comprising event data, at the processor, wherein the event data comprises a receipt image from a vendor associated with the event;
generating, by the processor, a unique identifier associated with the event data;
determining at the processor, at least one healthcare provider associated with the event; and
transmitting an email comprising the event data to at least one of: a healthcare provider associated with the event, an assistant to the healthcare provider, at least one representative, and the vendor associated with the event; and
storing, in the memory, in association with the unique identifier, the event data.

10. The system of claim 9, where in the receiving an event comprises receiving attendance data associated with the event data;

11. The system of claim 9, wherein determining the at least one healthcare provider comprises:
parsing the receipt image into time data and transaction data; and
assigning transaction data to the healthcare provider wherein the transaction data comprises: a vendor item; cost of the vendor item; and the healthcare provider associated with the vendor item.

12. The system of claim 11, further comprising generating a first-type alert based on the transaction data and transmitting the first-type alert to a representative manager or the at least one representative.

13. The system of claim 12, wherein generating the first-type alert comprises comparing the transaction data to a first threshold value associated with the at least one representative.

14. The system of claim 10, further comprising generating a second-type alert based on time data and the attendance data of the event.

15. The system of claim 14, wherein generating the second-type alert comprises:
comparing auxiliary time data and auxiliary attendance data of an auxiliary event with the time data and the attendance data of the event;
determining that a comparison of the time data and the attendance data exceeds a second threshold.

16. The system of claim 9, further comprising updating the event data based on receiving a response to the email transmitted to the healthcare provider.

17. A non-transitory computer-readable medium having computer-executable instructions encoded thereon that when executed, cause a computer to perform steps comprising:
receiving an event comprising event data, at a processor, wherein the event data comprises a receipt image from a vendor associated with the event;
generating, by the processor, a unique identifier associated with the event data;
determining at a processor, at least one healthcare provider associated with the event; and
transmitting an email comprising the event data to at least one of: a healthcare provider associated with the event, an assistant to the healthcare provider, at least one representative, and a vendor associated with the event; and
storing, in a memory, in association with the unique identifier, the event data.

18. The non-transitory computer-readable medium of claim 17, wherein determining the at least one healthcare provider comprises:
parsing the receipt image into time data and transaction data; and
assigning transaction data to the healthcare provider wherein the transaction data comprises: a vendor item; cost of the vendor item; and the healthcare provider associated with the vendor item.

19. The non-transitory computer-readable medium of claim 18, further comprising generating a first-type alert based on the transaction data and transmitting the first-type alert to a representative manager or the at least one representative.

20. The non-transitory computer-readable medium of claim 17, further comprising generating a second-type alert based on time data and attendance data of the event.
